(19) United States
(12) Patent Application Publication
(10) Pub. No.: US 2016/0051782 A1
(43) Pub. Date: Feb. 25, 2016

(54) Endotracheal Tube Bite Block

(71) Applicant: Rodney B. Reed, Antioch, CA (US)

(72) Inventor: Rodney B. Reed, Antioch, CA (US)

(21) Appl. No.: 14/463,282

(22) Filed: Aug. 19, 2014

(51) Int. Cl. A61M 16/04

(52) U.S. Cl. CPC A61M 16/0493

(57) ABSTRACT

The endotracheal tube bite block comprises two equivalent semi-circular elongated bodies partially connected to one another via linking tabs which enable the two semi-circular elongated bodies to move towards one another forming a circular elongated body to encapsulate the endotracheal tube. The endotracheal tube bite block has slope shaped design which will be placed inside the patient's mouth and rest between the teeth. The endotracheal tube bite block further comprises pressure tabs which prevent complete collapse of the device under extreme biting pressures.

### BACKGROUND OF THE INVENTION

1. Field of the Invention

This invention relates generally to endotracheal tube bite block, and more particularly to endotracheal tube sleeve that protects the endotracheal tube from occlusion.

2. Description of Related Art

Endotracheal tubes are intubated, i.e., inserted into the trachea, to provide a clear passage for air to the lungs for comatose patient. Also, an endotracheal may be intubated prior to anesthetizing a patient with a general anesthetic to maintain a clear air passage in case the trachea collapses.

Since the tubes are made of pliant material, a comatose patient may bite down on an oral endotracheal tube causing occlusion of the tube and constricting the air passage. A bite block may be inserted between the upper and lower teeth of the patient when an oral endotracheal tube is used.

Endotracheal tube bite blocks for providing clear air passage from mouth to the lung is known in the prior art. More specifically, by way of example, U.S. Pat. No. 4,527,559 to Roxburg et. al. discloses an elongated hard core which is encapsulated in a block of material not as hard as the core.

U.S. Pat. No. 4,166,467 to Abramson discloses an endotracheal tube assembly which includes a curved section of flexible tubing suitable for insertion into the trachea and having a junction fitting at its outer end extending outside of the mouth of the patient, the junction fitting providing a male connection having an interference fit with the tubing so that the tubing is stretched and enlarged in the region of overlap.

U.S. Pat. No. 3,946,742 to Eross discloses a bite block including a retainer having a slotted channel into which an oral endotracheal tube is inserted. The bite block is secured to the endotracheal tube by wrapping an elastic strap which is secured to the bite block around the endotracheal tube and engaging a selected hole in a strap with a post on the bite block, is wrapped around the neck and secured to a post on the bite block.

### SUMMARY OF THE INVENTION

The device is a bite block that is used with ventilator dependent patients that are endotracheal intubated and connected to a life support machine.

The device is used to protect the endotracheal tube from occlusion due to the biting pressure caused by the patient or any other obstruction that may cause a kink in the endotracheal tube that may prevent proper ventilation into the patients' lungs.

The endotracheal tube bite block comprises two equivalent semi-circular elongated bodies partially connected to one another via linking tabs which enable the two semi-circular elongated bodies to move towards one another forming a circular elongated body to encapsulate the endotracheal tube. The endotracheal tube bite block has slope shaped design which will be placed inside the patient's mouth and rest between the teeth. The endotracheal tube bite block further comprises pressure tabs which prevent complete collapse of the device under extreme biting pressures.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a top view showing the convex exterior surface of the two semi-circular elongated bodies of the device which is in an open position.

FIG. 2 is a concave interior surface of the two semi-circular elongated bodies of the device which is in an open position.

FIG. 3 shows an adhesive strip (tape) used to wrap around the device and the endotracheal tube.

FIG. 4 shows the device in a closed position wherein the two semi-circular elongated bodies surround the endotracheal tube and lock it in place.

### DESCRIPTION OF THE PREFERRED EMBODIMENT

The device is an endotracheal tube bite block that is used with ventilator dependent patients that are endotracheal intubated and connected to a life support machine.

The device is used to protect the endotracheal tube from occlusion due to the biting pressure caused by the patient or any other obstruction that may cause a kink in the endotracheal tube that may prevent proper ventilation into the patients' lungs.

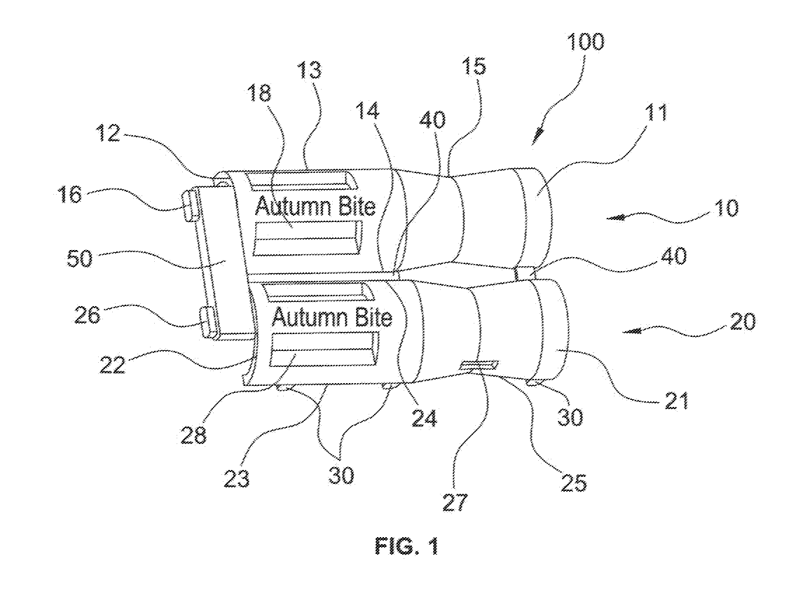

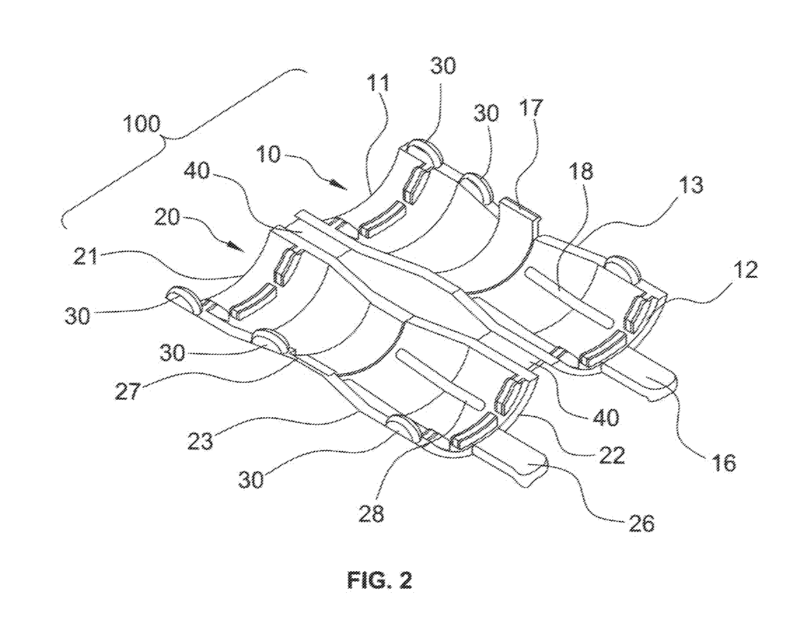

Referring to FIGS. 1-2, there is disclosed an endotracheal tube bite block 10 according to an embodiment of the present invention. FIGS. 1 and 2 shows the endotracheal tube bite block 10 in an opened position which will allow the healthcare worker to properly place the device 10 around the endotracheal tube for correct placement without disconnection of the ventilator circuit. FIG. 1 shows the convex exterior surface of the endotracheal tube bite block 100. FIG. 2 shows the concave interior surface of the endotracheal tube bite block 100.

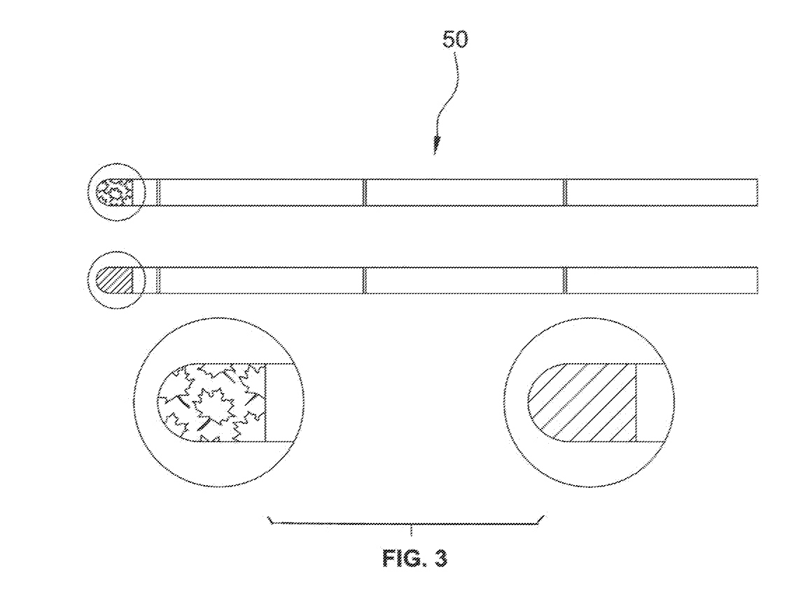

FIG. 3 shows an adhesive strip 50 used to wrap around the device 100 and the endotracheal tube 200.

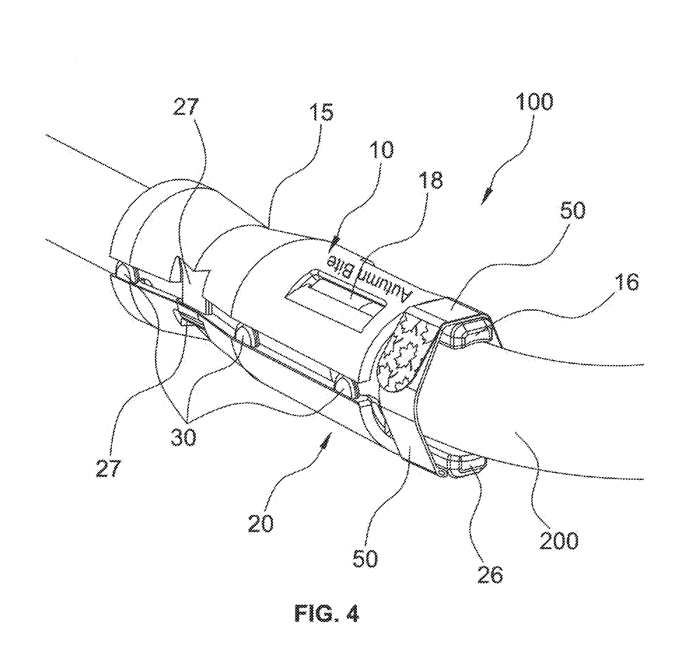

FIG. 4 shows the device 100 in a closed position wherein the endotracheal tube bite block 100 surrounds the endotracheal tube 200 and locks it in place.

Referring to FIGS. 1-4, the endotracheal tube bite block 100 comprises two semi-circular elongated bodies 10, 20 of the same shape and dimensions. Each elongated body 10, 20 has a front end 11, 21, a rear end 12, 22, and two edges 13, 14, 23, 24. The two edges of both elongated bodies have slope in and slope out 15, 25 shaped design. The slope shaped design 15, 25 will be placed inside the patient's mouth and rest between the teeth. The two elongated bodies 10, 20 are partially connected to one another on one of two edges 14, 24 near the front and rear ends via linking tabs 40 which enable the two semi-circular elongated bodies to move towards one another forming a circular elongated body to encapsulate the endotracheal tube. The endotracheal tube bite block 100 further has two protruding parts 16, 26, each connected to the rear end 12, 22 of each of the semi-circular elongated bodies.

The endotracheal tube bite block 100 further comprises a plurality of pressure tabs 30 which are connected to one of the semi-circular elongated bodies 20 on the edge 23 that are not connected to the other semi-circular elongated bodies. The pressure tabs 30 are in place to prevent complete collapse of the endotracheal tube bite block 100 under extreme biting pressure.

The endotracheal tube bite block 100 further comprises a locking tab 17 connected to one of the semi-circular elongated body 10 and a hole 27 located on the other semi-circular elongated body 20. The locking tab 17 snaps into the hole 27 to lock the two semi-circular elongated bodies into place in a closed position. With light pressure applied to the locking tab 17, the locking tab 17 can be released from the hole 27 whereby the device 10 can be re-opened.

The endotracheal tube bite block 100 may further comprise two open slots 18, 28, each located on each of the two semi-circular elongated bodies for the endotracheal securing strap to be placed through the open slots 18, 28 to secure the device to the endotracheal tube holder when the two semi-circular bodies 10, 20 are closed together around the endotracheal tube.

The endotracheal tube bite block 100 further comprises an adhesive tape 50 to wrap around the protruding parts 16, 26 of the device 100 and endotracheal tube to keep the device 100 in place once the device is in the closed and locked position. In the preferred embodiment, the adhesive strap is 167.27 (6.59) in length excluding the adhesive portion. The adhesive portion is 9.52 (0.375) in length. The width of the strap is 7 (0.28) in width. Graphic is added to one end of the adhesive tape 50 and no adhesive on bottom of graphic area.

In the preferred embodiment, the device is translucent in color with a light tint. Other color may be used in other embodiments.

In use, when the two semi-circular elongated bodies are placed around the endotracheal tube, the two semi-circular elongated bodies 10, 20 are closed towards one another and the lock tab 17 is snapped into the hole 27 to lock the two elongated bodies in place so that the device 100 can protect the endotracheal tube from occlusion caused by biting. The front end is placed inside the patient's mouth and the slope in and slope out shape design 15, 25 rests between the patient's teeth.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. A device for protecting an endotracheal tube in a patient's mouth from occlusion comprising:
two substantially semi-circular elongated bodies of same shape and dimensions, each semi-circular elongated body having a front end, a rear end, and two edges which have sloped shape design, the two semi-circular elongated bodies partially connected to one another on one of the two edges via linking tabs which enable the two semi-circular elongated bodies to move towards one another forming a circular elongated body to encapsulate the endotracheal tube;
two protruding parts each connected to the rear end of each semi-circular elongated body;
a plurality of pressure tabs connected to one semi-circular elongated body on the edge which is not connected to the other semi-circular elongated body, the pressure tabs being in place to prevent complete collapse of the device under extreme biting pressures; and
a hole located to one semi-circular elongated body and a locking tab connected on the other semi-circular elongated body to engage the hole, the locking tab snapped into the hole to lock the two semi-circular elongated bodies into place in a closed position;
wherein when the two semi-circular elongated bodies are placed around the endotracheal tube, the two semi-circular elongated bodies are closed towards one another to protect the endotracheal tube from occlusion caused by biting, the front ends of the semi-circular elongated bodies are placed inside the patient's mouth and the slope shape design rests between the patient's teeth when the device is in use.

2. The device of claim 1 further comprising an adhesive tape to wrap around the protruding parts of the device and endotracheal tube to keep the device in place once the device is in the closed and locked position.

3. The device of claim 2, wherein the locking tab is releasable from the hole by light pressure applied to the locking tab whereby unlocking the two semi-circular elongated bodies.

4. The device of claim 3 further comprising two open slots, each located on each of the two semi-circular elongated bodies for the endotracheal securing strap to be placed through the open slot to secure the device to the endotracheal tube holder.

5. The device of claim 4, wherein the adhesive strap is approximately 6.361 inches in length and 0.276 inches in width.
